(19) United States
(12) Patent Application Publication
(10) Pub. No.: US 2018/0132780 A1
(43) Pub. Date: May 17, 2018

(54) SYSTEMS AND METHODS OF MONITORING AND ENCOURAGING EXERCISE IN INFANTS

(71) Applicant: PARENTING SCIENCE LTD., Tel Aviv (IL)

(72) Inventor: Daniel SAAR, Tel Aviv (IL)

(21) Appl. No.: 15/537,419

(22) PCT Filed: Dec. 20, 2015
(86) PCT No.: PCT/IL2015/051229

§ 371 Date: Jun. 19, 2017

Related U.S. Application Data
(60) Provisional application No. 62/098,359, filed on Dec. 31, 2014.

(51) Int. Cl. A61B 5/00; A61B 5/11; A61B 3/113; A61B 3/117; A61B 5/024

(52) U.S. Cl. CPC A61B 5/486; A61B 2576/00; A61B 5/1114; A61B 5/1116; A61B 5/1118; A61B 5/1128; A61B 5/741; A61B 5/7425; A61B 5/743; A61B 5/7445; A61B 5/4833; A61B 3/113; A61B 5/6898; A61B 5/7246; A61B 5/1121; A61B 5/7282; A61B 3/1176; A61B 5/02405; A61B 5/445; A61B 5/746; A61B 5/0022; A61B 2560/0475; A61B 2503/04; A61B 2505/09; A61B 5/0077

(57) ABSTRACT

The present invention relates to systems and methods of monitoring and encouraging activity in infants, and more specifically, the present invention relates to systems and methods of interactively monitoring and encouraging infants to exercise their neck and back muscles. The present invention further comprises method of treating torticollis in infants.

### FIELD OF THE INVENTION

The present invention generally relates to monitoring and encouraging activity in infants, and specifically, the present invention relates to systems and methods of interactively monitoring and encouraging infants to exercise their neck and back muscles.

### BACKGROUND OF THE INVENTION

One of the first physical developmental steps infants need to accomplish is holding their head up while lying on their stomach. Infants need to lift their head in order to avoid spinal cord injuries, actively examine their surroundings, and to begin their motor preparation towards other developmental stages such as crawling and sitting. In order to be able to lift their head, infants need to strengthen their neck and back muscles. Infants gain this strength and improve their head lifting starting at birth, and usually up to the age of eight months. The major factor that determines the infant's progress in this task in the amount of time the infant practices postures that help him to strengthen his neck and back muscles. Specifically, the amount of time the baby lie down of his stomach, while he is awake, so he can practice head lifting, also known as “Tummy Time”.

Up until the beginning of the 90's, many parents have put their babies to sleep while lying on their stomach. As a result, Tummy Time was a natural position for infants and their neck and back strengthening exercises became a natural part of their routine. In the beginning of the 90's, due to a high correlation found between sudden infant death syndrome (SIDS) and sleeping while lying on the stomach, health authorities around the world have initiated a new recommendation for parents, which instructed parents to put their babies to sleep on their backs only. This recommendation was widely accepted by parents and changed the routine of infants around the world. This guidance had succeeded in reducing the SIDS rates among infants, but at the same time, intensified other medical conditions, such as head shape deformations and weak neck and back muscles, resulting in delays in motoric and physical development. Such motoric and physical developmental delays have been shown to be linked to other developmental delays, as perceptional, emotional and verbal skills.

As a result of these findings, the American Academy of Pediatrics and other health related governmental organization, issued an explicit recommendation to encourage Tummy Time exercise, in order to gain neck and back muscle strength. Therefore, parents are encourage to initiate Tummy Time by laying their babies on their stomach, while they are awake, surrounded by stimulations which should interest the baby, thus getting him to lift his head upwards. However, babies—at list initially—do not like being put to Tummy Times because it is physically difficult for them, and many times bores them due to the much less interesting stimulations their subjected to in comparison to lying on their backs. As a result, they get frustrated and start crying, causing many parents to finish this important exercise, to avoid the baby's crying. This unpleasant incident also causes many parents to postpone the initiation of new exercise sessions or to decrease the amount of Tummy Time exercise they initiate, resulting in further delays in strengthening of the neck and back muscles. In addition, currently there are no means for medical tracking of the exercise routine and gained muscle strength of babies, so both parents and medical professional, can't be alerted for the initial signs of developmental or medical problems caused by this situation.

It was recently found that approximately 46% of few-weeks old babies have some level of physiological medical condition related to head shape abnormality, attributed to the change in the sleeping habits of babies (reference is hereby made to The Changing Concept of Sudden Infant Death Syndrome: Diagnostic Coding Shifts, Controversies Regarding the Sleeping Environment, and New Variables to Consider in Reducing Risk, Pediatrics, Vol. 116 No. 5, 2005, pp. 1245-1255; Majnemer A, Dev Med Child Neurol. 2007 November; 49(11):804; Rosenberg S A et al., Pediatrics. 2008 June; 121(6):e1503-9; Pin T, Dev Med Child Neurol. 2007 November; 49(11):858-67; Majnemer A et al., J Pediatr. 2006 November; 149(5):623-629; and Reid M. V et al., Cog. Br. Beh. Vol IX(3), 193-200, 2005). Moreover, the above reference clearly mention a correlation between babies being laid to sleep on their back and motoric developmental delays, even well after babies acquire walking skills and even into their second year of age.

Furthermore, it was found that babies having more developed motoric skills understood human and social movements better than babies at the same age, with less developed motoric skills.

Additionally, Tummy Time exercise is used in treatment of pediatric torticollis. Torticollis, also known as twisted neck or wryneck, is a dystonic condition defined by an abnormal, asymmetrical head or neck position and is expressed by a fixed or dynamic tilt, rotation, or flexion of the head and/or neck. The incidence of torticollis is estimated to be 0.3-3.92% (Cheng, J C et al. The Journal of bone and joint surgery. American volume 83-A (5): 679-87; Chen M M, Chang H C, Hsieh C F, Yen M F, Chen T H. Arch Phys Med Rehabil. 2005 November; 86(11):2199-203). Since the early 90's when health authorities published recommendation to put babies to sleep on their back, the reported incidence of torticollis increases dramatically. This correlation may due to the fact that the recommendation decreases time infants spent on their tummy during the first months of their lives—the natural mechanism to correct congenital pediatric torticollis as well as to prevent acquired torticollis.

The treatment protocols for pediatric torticollis contain guiding the parents to promote head or neck symmetry by providing as many opportunities as possible for the baby to turn its head to the side that it normally doesn't turn to. It's also recommended for parents to give the baby plenty of Tummy Time exercise to in order to help strengthen their neck and back muscles.

Evidence can be found in the references above that the main reason for the consequences discussed above is the reduction in parent induced practices of Tummy Time and that Tummy Time exercise is beneficial for various aspects of baby's development. Therefore, there is a long-felt need for a method and means to effectively encourage infants to do their Tummy Time exercise as well as monitor their advancement in order to improve their health and/or prevent progression of medical conditions.

### SUMMARY OF THE INVENTION

Thus it is an object of the present invention to provide a method for monitoring and encouraging exercise of an infant, characterized by the steps of: a) positioning the infant in front of an optical sensor; b) displaying visual stimulation on a display means; c) recording engagement of said infant by the optical sensor; d) monitoring and analyzing in real-time the engagement of the infant by a processor, thereby obtaining performance information; e) storing the performance information on a storage means; and f) corresponding the visual stimulation to a protocol predetermined by selected features recognized in the performance information.

It is also an object of the present invention to provide the aforementioned method, wherein the step of recording and the step of monitoring and analyzing are carried out simultaneously.

It is also an object of the present invention to provide the aforementioned method, wherein the step of recording and the step of monitoring and analyzing start before the step of displaying visual stimulation.

It is also an object of the present invention to provide the aforementioned method further comprising the steps of: a) selecting the processor to be located in proximity to the optical sensor, or in a remote location; and b) selecting the storage means to be located in proximity to the optical sensor, or in a remote location.

It is also an object of the present invention to provide the aforementioned method further comprising the step of adjusting said visual stimulation when said performance information indicates condition consisting the group of: no engagement of the infant, the engagement of the infant for a predetermined period of time, progress of the infant, the infant's head, chest or hands in predefined postures or positions, a predefined gaze pattern, a predefined activity of the infant, a predefined reaction of the infant or any combination thereof.

It is also an object of the present invention to provide the aforementioned method further comprising the step of providing audio stimulation to accompany the visual stimulation, thereby providing audiovisual stimulation.

It is also an object of the present invention to provide the aforementioned method further comprising the step of altering said audiovisual stimulation when the performance information indicates condition consisting the group of: no engagement of the infant, the engagement of the infant for a predetermined period of time, progress of the infant, the infant's head, chest or hands in predefined postures or positions, a predefined gaze pattern, a predefined activity of the infant, a predefined reaction of the infant or any combination thereof.

It is also an object of the present invention to provide the aforementioned method further comprising the step of producing vocalizations when said performance information indicates condition consisting the group of no engagement of said infant, the engagement of the infant for a predetermined period of time, progress of the infant, the infant's head, chest or hands in predefined postures or positions, a predefined gaze pattern, a predefined activity of said infant, a predefined reaction of the infant or any combination thereof.

It is also an object of the present invention to provide the aforementioned method further comprising the step of altering the visual, audio, vocalization and/or audiovisual stimulation in accordance with parameters selected from the group consisting of the infant's age, the infant's performance information, the infant's progress, duration of a usage of the system, a user's choice of stimulation, a predefined diagnosed medical condition and any combination thereof.

It is also an object of the present invention to provide the aforementioned method further comprising the step of recommending the visual, audio, vocalization and/or audiovisual stimulation in accordance with parameters selected from the group consisting of the infant's age, the infant's performance information, the infant's progress, duration of a usage of the system, a user's choice of stimulation, a predefined diagnosed medical condition and any combination thereof.

It is also an object of the present invention to provide the aforementioned method further comprising the step of automatically stopping the stimulation after sensing an outcome selected from the group consisting of inability to detect face or gaze of the infant, losing tracking of the face or gaze of the infant, use of said stimulation for a predefined amount of time, a predefined activity of the infant, a predefined reaction of the infant and any combination thereof.

It is also an object of the present invention to provide the aforementioned method further comprising the step of stopping said stimulation manually.

It is also an object of the present invention to provide the aforementioned method wherein the display means is selected from the group consisting of a mobile phone, a tablet, a laptop, an e-reader, a digital wall, a smart TV, a personal computer, a wearable technology and any combination thereof.

It is also an object of the present invention to provide the aforementioned method further comprising the step of graphically presenting at least part of the performance information on a display means at the end of an exercise session.

It is also an object of the present invention to provide the aforementioned method further comprising the step of selecting features of the performance information from the group consisting of duration of an exercise session, height of the infant's face, highest height reached during exercise session, number of roll ups, duration of the engagement with the stimulation, comparison of current usage performance information with past usage performance information, comparison of current usage performance information with at least one other user's performance information, comparison of current usage performance information with cumulative peer group performance information, the infant's gaze correlation with the visual stimulation, the infant's physical steadiness, engagement steadiness, the amount of time spent in each position of the infant, the infant's reflected mood, the infant's hand position, body posture development level of the infant, the amount of time spent in each position of the infant's head, measured angle of the infant eyes related to the nose, measured angle of the infant eyes related to the horizon, information extracted from skin tone of the infant and any combination thereof.

It is also an object of the present invention to provide the aforementioned method further comprising the step of analyzing cumulative performance information at a predetermined frequency.

It is also an object of the present invention to provide the aforementioned method further comprising the step of detecting abnormal performance information relating to a medical conditions selected from the group consisting of abnormal or inability of the infant to track the visual stimulation, tendency of the infant to bias his engagement from a specific direction, significant deviance from the self-cumulative past performance information, significant deviance from the peer group-cumulative past performance information, abnormal orientation detection of said infant's head, strabismus, cataract, lazy eye, apathy, torticollis, abnormal orientation of the infant's body posture, hypotonia, hypertonia, anemia, skin rash, arrhythmia and any combination thereof.

It is also an object of the present invention to provide the aforementioned method further comprising the step of providing an alert following detection of the abnormal performance information.

It is also an object of the present invention to provide the aforementioned method wherein a predetermined time limit (e.g. 5 min) is imposed to a single exercise session.

It is also an object of the present invention to provide the aforementioned method wherein a predetermined time limit (e.g. 30 min) is imposed to daily exercise sessions.

It is also an object of the present invention to provide the aforementioned method wherein guidance is provided to position the infant correctly in front of the optical sensor and/or to position the optical sensor correctly in front of the infant.

It is also an object of the present invention to provide the aforementioned method wherein said guidance is selected from the group consisting of a silhouette of an infant, text instructions, visual instruction, audio instructions, color coded indicator and any combination thereof.

It is also an object of the present invention to provide the aforementioned method wherein said recording engagement results in video recording to said storage means at least part of an exercise session, thereby obtaining at least one archive video.

It is also an object of the present invention to provide the aforementioned method further comprising the step of choosing an exercise session to be archived or not by a user.

It is also an object of the present invention to provide the aforementioned method further comprising the step of processing at least part of said archive videos of the infant by an image processing element, selecting at least one peak performance image corresponding to at least one of said selected features recognized by said performance information and displaying the peak performance image on a display means.

It is also an object of the present invention to provide the aforementioned method further comprising the step of displaying archiving video and/or images of exercise session in a display means at the end of an exercise session.

It is also an object of the present invention to provide the aforementioned method further comprising the step of electronically sharing, with at least one other user of the display means, the content selected from the group consisting of archive videos, performance information, at least one peak performance image or any combination thereof.

It is also an object of the present invention to provide the aforementioned method, prior to the step of electronically sharing, further comprising the step of automatically or manually performing on the archive videos actions selected from the group consisting of editing, adding data, adding text, overlaying at least one visual feature, overlaying at least one audio feature, adding at least one peak performance image, editing a plurality of videos into a single video and any combination thereof.

It is also an object of the present invention to provide the aforementioned method further comprising digitally transmitting at least part of the video archive, the performance information and/or peak performance images to an electronic device selected from the group consisting of a remote server, a cloud-like server, a remote computer, a remote display device and any combination thereof, and optionally digitally transmitting from said electronic device, back to the display means, analyzed performance information, user instructions or any computer readable instructions pertaining to the performance information.

It is also an object of the present invention to provide the aforementioned method further comprising the step of transmitting at least part of the video archive, the performance information, and/or peak performance images to a medical facility or personnel, and optionally digitally transmitting from the medical facility or personnel, back to said display device, analyzed performance information, user instructions or any computer readable instructions pertaining to the performance information.

It is also an object of the present invention to provide the aforementioned method wherein said medical facility is selected from the group consisting of a family doctor, a pediatrics doctor, an orthopedic, a neurologist, an ophthalmologist, a physiotherapist, a nurse, a healthcare personnel, a medical information center, a telehealth or telemedicine service and any combination thereof.

It is also an object of the present invention to disclose a computer executable program adapted to perform aforementioned method when running on a computer, a network or a server system.

It is also an object of the present invention to disclose a computer-readable storage medium, having encoded thereon the aforementioned computer program.

It is another object of the present invention to provide a system for monitoring and encouraging exercise of an infant comprising: a) an optical sensor configured to detect and record engagement of said infant; b) a processor configured to monitor and analyze in real-time the engagement of the infant for obtaining performance information; c) a storage means for storing the performance information and d) a display means. Wherein the processor is configured to cause said display means to display visual stimulation corresponding to a protocol predetermined by selected features recognized by said performance information.

It is also an object of the present invention to provide the system as defined in any of above, wherein the processor is located in proximity to said optical sensor, or in a remote location.

It is also an object of the present invention to provide the system as defined in any of above, wherein the storage means is located in proximity to said optical sensor, or in a remote location.

It is also an object of the present invention to provide the system as defined in any of above, wherein said visual stimulation is altered when said performance information indicates condition consisting the group of no engagement of the infant, the engagement of the infant for a predetermined period of time, the infant's head, chest or hands in predefined postures or positions, a predefined gaze pattern, a predefined activity of the infant, a predefined reaction of the infant, the progress of the infant or any combination thereof.

It is also an object of the present invention to provide the system as defined in any of above, wherein the system further comprises audio means.

It is also an object of the present invention to provide the system as defined in any of above, wherein the processor is configured to cause the audio means to generate music to accompany the visual stimulation, for providing audiovisual stimulation.

It is also an object of the present invention to provide the system as defined in any of above, wherein the audiovisual stimulation is altered when the performance information indicates condition consisting the group of no engagement of the infant, the engagement of the infant for a predetermined period of time, progress of the infant, the infant's head, chest or hands in predefined postures or positions, a predefined gaze pattern, a predefined activity of the infant, a predefined reaction of the infant or any combination thereof.

It is also an object of the present invention to provide the system as defined in any of above, wherein the processor is configured to cause the audio means to produce vocalizations when said performance information indicates condition consisting the group of no engagement of said infant, the engagement of said infant for a predetermined period of time, progress of the infant, the infant's head, chest or hands in predefined postures or positions, a predefined gaze pattern, a predefined activity of the infant, a predefined reaction of the infant or any combination thereof.

It is also an object of the present invention to provide the system as defined in any of above, wherein the visual, audio, audiovisual stimulation and vocalization is altered in accordance to parameters selected from the group consisting of the infant's age, the infant's performance information, the infant's progress, duration of a usage of the system, a user's choice, a predefined diagnosed medical condition and any combination thereof.

It is also an object of the present invention to provide the system as defined in any of above, wherein said system recommends the visual, audio, audiovisual stimulation and vocalization in accordance with parameters selected from the group consisting of the infant's age, the infant's performance information, the infant's progress, duration of a usage of the system, a user's choice of stimulation, a predefined diagnosed medical condition and any combination thereof.

It is also an object of the present invention to provide the system as defined in any of above, wherein the processor is configured to automatically stop the stimulation after sensing an outcome selected from the group consisting of inability to detect face or gaze of the infant, losing tracking of the face or gaze of the infant, use of the stimulation for a predefined amount of time, a predefined reaction of the infant and any combination thereof.

It is also an object of the present invention to provide the system as defined in any of above, wherein the stimulation is stopped manually by a user.

It is also an object of the present invention to provide the system as defined in any of above, wherein the processor is configured to graphically present at least part of the performance information, archive video and/or peak performance images on the display means at the end of an exercise session.

It is also an object of the present invention to provide the system as defined in any of above, wherein the display means is selected from the group consisting of a mobile phone, a tablet, a laptop, an e-reader, a smart TV and a personal computer, a digital wall, a wearable technology and any combination thereof.

It is also an object of the present invention to provide the system as defined in any of above, wherein guidance is provided for a user to position the infant correctly in front of the optical sensor and/or to position the optical sensor correctly in front of the infant.

It is also an object of the present invention to provide the system as defined in any of above, wherein said guidance is selected from the group consisting of a silhouette of an infant, text instructions, visual instruction, audio instructions, color coded indicator and any combination thereof.

It is also an object of the present invention to provide the system as defined in any of above, wherein a predetermined time limit (e.g. 5 min) is imposed to a single exercise session.

It is also an object of the present invention to provide the system as defined in any of above, wherein a predetermined time limit (e.g. 30 min) is imposed to daily exercise sessions.

It is also an object of the present invention to provide the system as defined in any of above, wherein a user chooses video and performance information of an exercise session to be archived or not.

It is also an object of the present invention to provide the system as defined in any of above, wherein the selected features recognized by the performance information are selected from the group consisting of duration of exercise session, height of the infant's face, highest height reached during exercise, number of roll ups, duration of said engagement with the stimulation, comparison of current usage performance information with past usage performance information, comparison of current usage performance information with at least one other user's performance information, comparison of current usage performance information with cumulative self or peer group's past usage performance information, the infant's gaze correlation with the visual stimulation, engagement steadiness, the amount of time spent in each position of the infant, the infant's reflected mood, the infant's hand position, body posture development level of the infant, the amount of time spent in each position of the infant's head, measured angle of the infant eyes related to the nose, measured angle of the infant eyes related to the horizon, information extracted from skin tone of the infant (for indication of skin problem, heart rate, anemia et al) and any combination thereof.

It is also an object of the present invention to provide the system as defined in any of above, wherein the processor is further configured to analyze cumulative performance information at a predetermined frequency.

It is also an object of the present invention to provide the system as defined in any of above, wherein the processor is configured to detect abnormal performance information relating to a performance selected from the group consisting of abnormal or inability of said infant to track said visual stimulation, tendency of said infant to bias his engagement from a specific direction, significant deviance from said self-cumulative past performance information, significant deviance from the peer group-cumulative past performance information, abnormal orientation detection of said infant's head, strabismus, cataract, lazy eye, apathy, torticollis, abnormal orientation of said infant's body posture, hypotonia, hypertonia, anemia, skin rash, arrhythmia and any combination thereof.

It is also an object of the present invention to provide the system as defined in any of above, wherein the processor is configured to provide an alert following detection of the abnormal performance information.

It is also an object of the present invention to provide the system as defined in any of above, wherein said storage means is configured to record the engagement in at least part of an exercise session resulting in at least one archive video.

It is also an object of the present invention to provide the system as defined in any of above, further comprising an image processing element configured to process capturing of the engagement of the infant, select at least one peak performance image corresponding to at least one of the selected features recognized by the performance information and display at least one said peak performance image on the display means.

It is also an object of the present invention to provide the system as defined in any of above, wherein the system is configured to encourage a user to electronically share with at least one other user of the display means the group consisting of archive videos, performance information, at least one peak performance image and any combination thereof.

It is also an object of the present invention to provide the system as defined in any of above, wherein the processor is configured to automatically or manually perform on the archive videos action consisting the group of editing, adding data, adding text, overlaying at least one visual feature, overlaying at least one audio feature, adding at least one peak performance image, editing a plurality of videos into a single video and any combination thereof.

It is also an object of the present invention to provide the system as defined in any of above, further comprising communication means configured to electronically transmit at least part of the performance information, the archive video and/or peak performance images to an electronic device selected from the group consisting of a remote server, a cloud-like server, a remote computer, a remote display device and any combination thereof, and optionally the electronic device comprises communication means configured to electronically transmit back to the device analyzed performance information, user instructions or any computer readable instructions pertaining to the performance information.

It is also an object of the present invention to provide the system as defined in any of above, wherein the electronic device is located in a medical facility, and optionally said medical facility comprises communication means configured to electronically transmit back to said device analyzed performance information, user instructions or any computer readable instructions pertaining to said performance information.

It is also an object of the present invention to provide the system as defined in any of above, wherein the medical facility is selected from the group consisting of a family doctor, a pediatrics doctor, an orthopedic, a neurologist, an ophthalmologist, a physiotherapist, a nurse, a healthcare personnel, a medical information center, a telehealth or telemedicine service and any combination thereof.

It is another object of the present invention to provide non-transitory, tangible, computer-readable storage media containing a program of instruction containing algorithms configured to cause a computer processing system running the program of instructions to monitor and encourage exercise of an infant by: a) providing guidance to correctly positioning the infant relative to an optical sensor; b) displaying visual stimulation; c) recording engagement of the infant by the optical sensor; d) monitoring and analyzing in real-time said engagement of the infant by the processor, thereby obtaining performance information; e) storing the performance information on a storage means; and f) corresponding the visual stimulation to a protocol. Wherein said protocol is predetermined and the stimulation is altered according to selected features recognized in the performance information.

It is another object of the present invention to provide a method of treating torticollis in an infant, the method is characterized by the steps of: a) analyzing performance information from at least one exercise session of said infant; b) detecting torticollis pattern in the infant; c) establishing quantitative baseline of said infant's medical symptom based on analysis of said at least one exercise session; d) selecting a treatment protocol from a preset database of stimulation for said infants according to at least one parameter of said infant; and e) tracking treatment progress of the infants.

It is still another object of the present invention to disclose the above mentioned method of treating torticollis, wherein the at least one parameter of the infant is selected from the group consisting of the infant's age, the medical symptom of the infant, the infant's performance information, a healthcare personnel's decision, a user's choice, the infant's treatment progress and any combination thereof.

It is still another object of the present invention to disclose the above mentioned method of treating torticollis, wherein the medical symptoms of the infant is selected from the group consisting of type of torticollis, amplitude of tilt, amplitude of rotation, amplitude of flexion and any combination thereof.

It is still another object of the present invention to disclose the above mentioned method of treating torticollis, further comprising the step of alerting diagnosis of torticollis in said infant to relevant healthcare personnel after said step of detecting torticollis pattern.

It is still another object of the present invention to disclose the above mentioned method of treating torticollis, wherein diagnosis of torticollis is performed by a healthcare personnel.

It is still another object of the present invention to disclose the above mentioned method of treating torticollis, wherein the healthcare personnel is selected from the group consisting of a physician, a nurse, a physician assistant, a nursing assistant, an attendant, a technician, a physiotherapist, a contractual staff not employed by a health-care facility, a telemedicine service and any combination thereof.

It is still another object of the present invention to disclose the above mentioned method of treating torticollis, wherein said preset database of stimulation comprises a series of visual stimulation.

It is still another object of the present invention to disclose the above mentioned method of treating torticollis, further comprising the step of providing audio stimulation to accompany the visual stimulation, thereby providing audiovisual stimulation.

It is still another object of the present invention to disclose the above mentioned method of treating torticollis, further comprising the step of providing vocalization as a form of stimulation.

It is still another object of the present invention to disclose the above mentioned method of treating torticollis, wherein the visual, audio, audiovisual stimulation or vocalization is designed to adjust focus and/or content of the stimulation in a display means according to the at least one parameter of the infant.

It is still another object of the present invention to disclose the above mentioned method of treating torticollis, further comprising the step of manually changing treatment protocol by a healthcare personnel.

It is still another object of the present invention to disclose the above mentioned method of treating torticollis, wherein said method promotes parent of said infant to compliant with daily exercise requirement.

It is still another object of the present invention to disclose the above mentioned method of treating torticollis, further comprising the step of digitally transmitting the performance information, archived videos and peak performance images from exercise sessions and/or treatment progress of the infant to an electronic device selected from the group consisting of a server, a network, a cloud-like server, a computer, a display means and any combination thereof, and optionally digitally transmitting, from the electronic device back to the display means, analyzed performance information, user instructions or any computer readable instructions pertaining to the performance information.

It is still another object of the present invention to disclose the above mentioned method of treating torticollis, further comprising the step of remotely accessing the data of exercise sessions and/or the treatment progress by a healthcare personnel.

It is still another object of the present invention to disclose the above mentioned method of treating torticollis, further comprising the step of sending automatic update of treatment progress of the infant to relevant healthcare personnel or at least one family member.

### BRIEF DESCRIPTION OF THE DRAWINGS

In order to understand the invention and to see how it may be implemented in practice, several embodiments will now be described, by way of non-limiting example only, with reference to the accompanying drawing, in which:

FIG. 1 schematically presents a high level overview of various embodiments of the systems and methods disclosed in the present invention.

FIG. 2 schematically presents a high level flow chart of a preferred embodiment of the system disclosed in the present invention.

FIG. 3 presents an embodiment of a high level overview of the method recited in the present invention.

FIG. 4 schematically presents an overview of an exercise session according to a preferred embodiment of the systems and methods disclosed in the present invention.

FIG. 5 schematically presents a high level flow chart of a preferred embodiment of the method of treating torticollis disclosed in the present invention.

FIG. 6 presents statistical results (n=36) of impact of using the disclosed system in the present invention to encourage exercise in infants; 6a shows the result of improvement in average number of daily exercise sessions and 6b shows the result of improvement in average session duration.

### DETAILED DESCRIPTION OF THE PREFERRED EMBODIMENTS

The following description is provided, alongside all chapters of the present invention, so that to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a system for monitoring and encouraging exercise of an infant, comprising an optical sensor configured to detect and track face position and orientation and gaze of the infant with respect to a display device; a processor configured to monitor and analyze in real-time the face position and orientation and gaze of the infant for obtaining performance information; a storage means for storing the performance information; and a display means; wherein the processor is configured to cause the display means to display visual stimulation corresponding to a protocol predetermined by selected features recognized by the analysis of the performance information. Furthermore, the present invention provides a method and system for treating torticollis in an infant.

The term ‘infant’ refers hereinafter to a baby in the age between birth and up until learning to walk, typically at the age of 18 months. The terms infant and baby are used interchangeably throughout this document.

The term ‘exercise’ refers hereinafter to an activity or position of an infant where active engagement of the neck and back muscles and surrounding muscles is present.

The term ‘user’ refers hereinafter to a person operating the method or system provided in the present application, and may refer to a parent or caregiver of the infant, or any healthcare personnel.

The term ‘healthcare personnel’ refers hereinafter to a person involving in healthcare of an infant. Examples include but not limit to a physician, a nurse, a physician assistant, a nursing assistant, an attendant, a technician, a physiotherapist a contractual staff not employed by a health-care facility.

The term ‘processor’ refers hereinafter to a hardware configured to execute a program of instructions.

The term ‘optical sensor’ refers hereinafter to a camera which can capture images, video, etc., or any other type of optical instrument configured for image sensing by converting light into electrons, such as a charge-coupled device (CCD) or complementary metal-oxide-semiconductor (CMOS). The various modes of operation of the sensor can be controlled by the processor settings. The sensor settings can include exposure, sensitivity, gain, noise, backlight, etc.

The term ‘storage means’ refers hereinafter to any electronic data storing device. Examples include but not limit to disc, hard drive, flash drive, memory stick, cloud service et al.

The term ‘display means’ refers hereinafter to any electronic display feature or device. Examples include but not limit to a mobile phone, a tablet, a computer, an e-reader, a digital wall, a smart TV, a wearable technology and a monitor.

The term ‘face orientation’ refers hereinafter to the height, direction and angular position of the head of an infant with respect to a defined coordinate frame or with respect to the ground upon which the infant is lying, or with respect to a display device the infant is looking at.

The term ‘gaze’ refers hereinafter to where a person is looking, as well as to tracking or monitoring where a person is looking. Gaze tracking may include tracking both the orientation and location of one eye or both eyes with respect to a defined coordinate frame. Hereafter, it will be understood that the term “eye”, includes one eye or both eyes of a subject.

The term ‘engagement’ refers hereinafter to the status of an infant with respect to a display device. The infant is considered to be engaged when his face orientation is such that the infant is directing his face to the display device, and/or lifting his head up from the floor, and/or when the infant's gaze is detected to be looking at the display device. The infant is considered to be not engaged when looking away from the display device, and/or lowering his head or gaze to the floor.

The term ‘performance information’ refers hereinafter to raw data recorded by the optical sensor as well as data and parameters gained by analyzing the raw data derived from the tracking of an infant's face orientation and gaze, such as in a non-limiting example, height and angle of the infant's face, the infant's body posture, height, direction and orientation of the infant's face and/or gaze, duration of the engagement with the stimulation, comparison of current usage performance information with past usage performance information, comparison of current usage performance information with at least one other user's performance information, duration of exercise session, infant's steadiness (i.e. how varied is the variance across a set of data), infant's body and head posture, number of roll ups, highest point of head, number of exercise sessions taken in a predetermined period of time, progress of exercise over time (i.e. trend of the data), engagement of gaze, eye tracking, flash reflection of the eyes, infant's muscle tension (hypotonia, hypertonia), infant's skin color changes, infant's heart rhythms, strabismus detection, cataract detection, lazy eye detection, the amount of time the infant spends in each position, the infant's reflected mood, infant's facial expressions, measured angle of the infant eyes related to the nose, measured angle of the infant eyes related to the horizon, and the like. The angle of the infant eyes related to the nose and/or horizon is measured by creating a virtual line on the display means that connects the infant's eyes and examining the virtual line in relation to the nose and/or the horizon.

The term ‘visual stimulation’ refers hereinafter to any digitally transmitted visual animation, image or video. In the present invention, visual stimulation is designed to attract an infant's attention. According to some preferred embodiment, visual stimulation comprises at least one animated animal figure with a large face with contrast colors.

The term ‘audio stimulation’ refers hereinafter to any digitally transmitted audio voice, sound or music.

The term ‘vocalization’ refers hereinafter to any sound with voice. It can be prerecorded or customized according to a user's choice.

The term ‘audiovisual stimulation’ refers hereinafter to any digitally transmitted audio stimulation along with visual stimulation.

The term ‘enhance’ refers hereinafter to an increase with respect to a current state. It is used interchangeably with “progress” throughout the document.

The term ‘alter’ refers hereinafter to the act of making any change to stimulation, treatment protocol et al. It is used interchangeably with “adjust” throughout the document.

The term ‘exercise session’ refers hereinafter to complete or partial exercise routine employed by the system and methods disclosed in the present invention.

The term ‘computer’ refers hereinafter to any device that can be programmed to carry out a set of arithmetic or logical operations automatically, including a personal computer, a mobile phone, a tablet, a notebook, a laptop, an e-reader, a smart television, a smart wall and the like.

The term ‘communication means’ generally refers hereinafter to any electronic circuitry and corresponding software that can be used to enable a device to communicate with a remote location, either by wireline or wirelessly.

The term ‘abnormal’, especially in the context of ‘abnormal performance information’, refers hereinafter to any observational difference in the development and behavior of the infant when compared to either his previous records or his peer group's average characteristics, including not raising the head, not raising the head for periods long enough, raising the head in abnormal orientation, body posture, organs shape abnormality, not gazing in a proper manner (such as in strabismus, lazy eye, autism, meningitis), inability to track objects, infant's muscle tension abnormality (hypotonia, hypertonia), infant's skin color abnormalities (Anemia, Skin rash and diseases), abnormal heart rhythms (arrhythmia), mood and expression abnormality (such as constant apathy) fever disease, dehydration, detecting flash reflection which indicates cataract and the like.

The term ‘medical diagnostic alert’ refers hereinafter to any visual or textual or audible stimulation that appears on the device of the user or in a medical personnel device indicating there is an abnormal performance information of the infant, indicative in the analysis of the performance information.

The term ‘image’ refers hereinafter to any digital image, photo, animation or video.

The term ‘peak performance image’ refers hereinafter to an image that exhibits the infant in the most favorable features, such as best image contrast, best image focus, highest face orientation, fully engaged gaze and the like.

The term ‘overall progress’ refers hereinafter to a parameter of a weighted average of performance information.

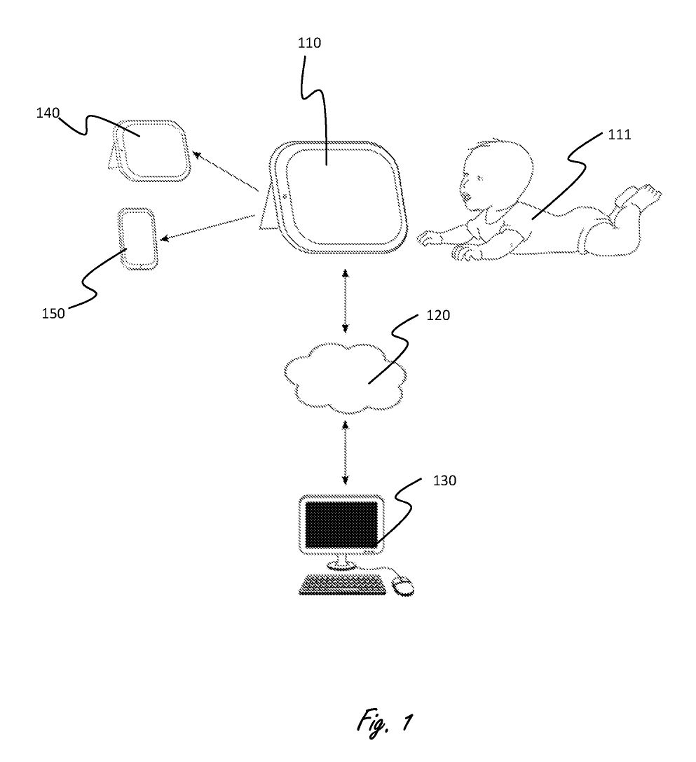

Reference is now made to FIG. 1, schematically illustrating a high level overview of preferred embodiments of the systems and methods disclosed by the present invention. The present invention discloses an interactive stimulation system for infants, for encouraging the infants to exercise their back and neck muscles and/or monitoring their progress. The method disclosed by the present invention recites tracking engagement of infant 111 with display screen 110, and interactively altering the audiovisual stimulation induced by the display device, in real-time, in accordance with the infant's engagement. The infant's engagement is determined by analysis of the raw data received by tracking the infant, resulting in defining the position and/or direction of the infant's face and/or gaze, performance information which could be, but not limited to, the duration of the exercise session, the height of the infant's face, duration of the engagement with the stimulation, comparison of current usage performance information with past usage performance information, comparison of current usage performance information with at least one other user's performance information, infant's body posture, infant's head position, infant's muscle tension (hypotonia, hypertonia), infant's skin color changes, infant's facial expression, the infant's gaze tracking correlation with the visual stimulation, engagement steadiness, the amount of time spent in each position of the infant, the infant's reflected mood (such as smiling, crying), the infant's hand position, strabismus detection, cataract detection, lazy eye detection, and any combination thereof. Alteration of the visual and/or audio stimulation is executed according to predetermined protocols corresponding to any selected features of the performance information. According to a preferred embodiment, the present invention disclosed a computer executable program adapted to encourage and monitor the infant's exercise of back and neck muscles when running on a computer (e.g. tablets, a laptop, a smart television, a smart wall, a wearable technology etc), a network or a server system.

According to a preferred embodiment of the present invention, display screen 110 shows a user guiding information on how to position the infant in front of the optical sensor correctly and/or how to position the optical sensor relative to the infant. The guiding information includes but not limits to a baby silhouette, an indication frame, text or visual instructions, a color coding image indicating the accuracy of the position. Only when the optical sensor is correctly positioned relative to the infant, the exercise session can begin.

In certain embodiments of the present invention the raw data received by tracking the infant's engagement is analyzed by a processor in display device 110. In other embodiments the raw data is transmitted to server 120 and analyzed by a processor there. Server 120 may be a server, a cloud-like server or a computer station located remotely from display device 110. Server 120 may contain storage means in the form of computer readable medium and a processor. Sever 120 may serve as a main computing center, collecting, storing and analyzing data from all users of the systems and methods recited in the present invention. This main computing center executes various calculations based on the collective data received by each display device 110, and outputs statistics relating to the usage of the method, as well as statistics of the performance information collected from the users of the method. These statistics may include any averages or standard deviation analysis pertaining to the performance information collected from the various users. Such statistics may be further transmitted from server 120 back to display device 110, or could be further transmitted to third computer center 130. In some embodiments, server 120 may analyze the raw data received by display device 110 pertaining to the engagement of the infant, assess the data and obtain performance information, and according to predetermined protocols send instructions back to the display device for altering the visual and/or audio stimulations. Server 120 may be wirelessly connected to display device 110, or connected through a wired connection.

In yet other embodiments the raw data is transmitted directly to computer 130 and may be analyzed, or further analyzed there. Computer 130 is any computer, computer station or server being used by third party of interest, other than the user of the device or the distributor of the method, such as, but not limited to, medical centers, a telehealth/telemedicine service, educational centers, research centers, a family doctor, a pediatrics, an orthopedic doctor, a neurologist, an ophthalmologist, a physiotherapist, a nurse or any other health professional and the like. Tracking of the infant may be analyzed for understanding usage routines and habits as well as to look for abnormal developmental progress such as abnormal or inability of the infant to track the visual stimulation, tendency of the infant to bias his engagement from a specific direction (suggesting torticollis condition), abnormal orientation detection of the infant's head or body, as well as to look for other medical conditions such as cataract, lazy eye, strabismus, autism, meningitis, hypotonia, hypertonia, infant's skin color and abnormalities (Anemia, Skin rash and diseases), abnormal heart rhythms (arrhythmia) and the like.

In some preferred embodiments, the raw data, in the form of video and/or images, is recorded and stored in the storage means of display device 110. The entire video recording of the exercise session is recorded in the storage means resulting in archive video, which is available to access from the device features and from the application itself. The videos are recorded to and stored in the picture/video standard gallery of the display device or in the application within the devise or transmitted to be stored in a secure cloud storage device, and are available for editing, sharing, uploading and overlaying any other visual/audio/data on them. Visual or audio overlays may include, but are not limited to, image borders, animations, text balloons, text boxes, music, voicing of the user, text relating to performance information, graphs and statistics of performance information analysis and the like. A plurality of videos can be edited to one video. Maneuvering video or images, such as editing, sharing, uploading or overlaying can be carried out automatically or manually. The peak performance data is saved in a first come-first out manner, preferably saving a slot for 5-20 images. This data, as well as the archived video and the analyzed performance information data, may be digitally shared with other electronic devices 140 and 150, which could be, but are not limited to a mobile phone, a tablet, a laptop, a smart TV, a digital wall, a personal computer, a wearable technology and any other display device with electronic communication means, transmitted wirelessly or wired. Digitally sharing this content with at least one other user of a display device may include indirect sharing by uploading the content to websites or social sharing platforms and the like. In an embodiment, the video, image and/or data may be sent with an overlay of animations and/or sounds, such as narrative commentary, border animations and the like. More in preferred embodiments, analysis of the raw data results in at least one peak performance image, where the infant is imaged raising his head high and preferably gazing into the device. This image may be displayed on the display device at the end of a session of exercise.

According to some preferred embodiments, after an exercise session is performed and recorded, a user can choose between a “baby session”, in which the exercise session video and related performance information is stored in the storage means, or a “test session”, in which the exercise session video and related performance information is not archived or stored in the computer executable program for further analysis. The “test session” video may still be stored in the storage means (e.g. in video gallery), but it cannot be accessed by the computer executable program.

According to some preferred embodiments, a predetermined time limit is imposed for single use and daily use of the systems and methods disclosed in the present invention. For example, a single exercise session is limited to 5 min and daily quota is limited to 30 min total. This is to avoid over exercise and to prevent collecting false data.

The stimulation provided by the display device relates to visual stimulation which is preferably accompanied by audio stimulation. The visual stimulation may be any sequence of images, or any videos of objects which are easily tracked by eye, preferably high contrast images, more preferably animated images. Animations may be provided either in black and white, black and white and red, or colorful. Alteration of the visual stimulation may be executed by changing the type of the content, enlarging the animation, providing multiple animation characters, increasing the speed and/or direction and/or variety of the movement of the animation characters, enhancing the colors and the like.

Audio stimulation may be given in two manners: (1) music, or soundtrack to accompany the visual stimulation; and (2) vocalizations, such as short readouts or calls, words, sentences or calling for attention expressions. The first stimulation which is music may be altered by changing the sound track, increasing the sound of the music, and also by increasing its tempo, or introducing at least one more attention grabbing instruments, such as in a non-limiting example, drums, trumpets and the like. The music may also be altered to return to the baseline. Vocalizations may be used to encourage an infant after detecting full engagement (relating to high head raising, good gaze tracking, long periods of engagement, the changing of the displayed content, etc.) with encouraging calls, or they may be used to gain lost attention of the infant by attention grabbing voices. The second stimulation could be preset or be customized such as recorded voice of the parents. It can also accompany the visual and/or audio stimulation according to a predetermined protocol.

Predetermined protocols relate to various outcomes in the visual and/or audio stimulation provided by device 110, corresponding to various behavior patterns of the infant detected by the engagement tracking. In a preferred embodiment, the stimulation provided by display device is adapted to alter whenever there is indication that the infant is looking away, losing interest, lowering his head, rolling away from their position, or when a full engagement of the infant is detected for more than a predetermined period of time. The interaction algorithm adapts the stimulation presented according to four different levels: adaptation of the video content to the developmental stage of the infant (e.g. age) or the infant's diagnosed medical condition (e.g. torticollis), adaptation of the visual stimulation according to the face position and/or orientation, gaze direction and/or session duration, adaptation of the audio stimulation according to the content type, face position and/or orientation, gaze direction and/or session duration and adaptation of the vocal stimulation accompanying the visual and audible stimulations based on the infant's face position, face orientation, gaze direction, gaze location, posture, session's progress, performance and the time elapsed since last stimulation.

In preferred embodiments, analysis of the performance information may take into consideration other general parameters relating to the infant and the practice, such as in a non-limiting example, infant's age, sex, daily hour of practice, content type of each practice segment, exercise routines, rate of progress, geographic location, user's language, location weather, etc.

Engagement of the infant is executed by any algorithm of face recognition and tracking and gaze tracking. The algorithm for the face orientation and tracking and gaze tracking may be, in a non-limiting example, algorithms based on modified OPENCV files, specifically adapted for infants. Adaptations may include skin color screening, pupil identification and pupil reflection tracking, consideration of the infant's repositioning by the parent, consideration of display device 110's position (vertically positioned or horizontally, etc.).

The program of the present invention is designed to execute both real-time adaptation of the visual and/or audible stimulation based on the infant's progress and performance information and at the same time record and document the infant's current progress and performance information. Moreover, the present invention provides both real-time tracking as well as video recording, which require further optimization of the hardware features of currently available computers and digital devices to enable two such types of input data through the same optical sensor.

The performance information is accumulated during the exercise session. The current exercise session's performance information is subjected to various calculations which are also compared to previous performance information from previous sessions, as well as to cumulative performance information collected from the infant's peer group. The results of the input analysis may be stored in a digital storage means, presented in the display or subjected to further medical assessment. The medical assessment compares results of the performance information to past performance data, known developmental milestones and to cumulative physical performance collected by the systems and methods of the present invention via multiple display devices, in order to identify any abnormal performance information.

According to a preferred embodiment, index of progress is calculated according to exercise sessions' performance information. In particular, overall progress of the infant is provided, which includes weighted average of relevant parameters such as exercise time, number of roll ups, highest height reached, the infant's gaze correlating with the stimulation, duration of the engagement with the stimulation, infant's head, chest and hand postures or positions, time spent in each posture or position, the infant's physical steadiness, the infant's engagement steadiness et al.

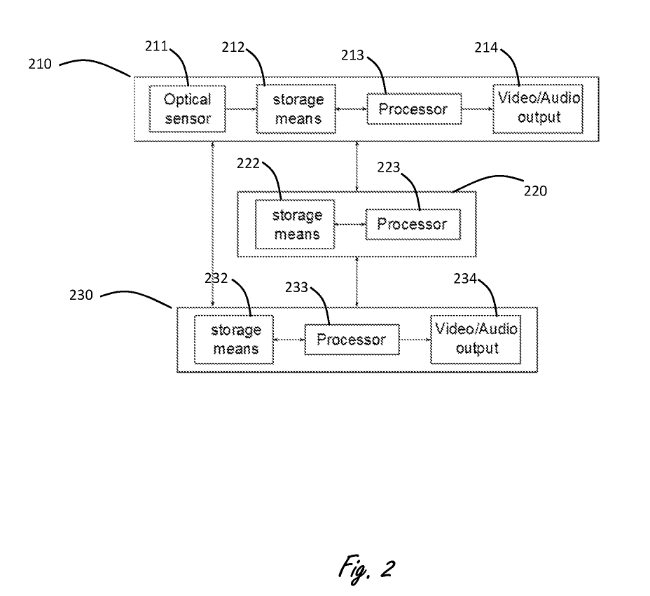

Reference is now made to FIG. 2, illustrating a high level overview of the system provided in the present invention. The present invention provides the system to encourage exercise in infants and/or monitor his progress, providing stimulations in accordance with the infant's engagement. The system is provided in display device 210, comprising optical sensor 211. In an embodiment, display device 210 further comprises storage means 212, processor 213 and a video and audio output means 214. The optical sensor 211 is configured to detect the face and gaze orientation, location and position with respect to display device 210. The recording of the infant, performed by the optical sensor, provides raw data in the form of videos, images and location coordinates, which are preferably saved in storage means 212. In the meanwhile, processor 213 is configured to analyze and monitor a confidence level of the face and/or gaze identification in real time. According to the confidence level calculation, the processor is configured to decide whether a session can continue tracking the identified coordinates representing these elements or a new attempt for facial recognition will be initiated. Processor 213 may also perform analysis of the raw data resulting in performance information. In some embodiments, the optical sensor and processor may also be configured to detect the hands position of the infant and to subject this data into further analysis. The progress of the infant may be analyzed for each session independently, or it can be analyzed with respect to any number of sessions previously saved in storage means 212 in display device 210. According to this embodiment, the application is ‘stand-alone’ and does not require any communication with external sources to complete its analysis.

According to preferred embodiments, a summary of at least part of the analysis and results of provided by the performance information is be presented through images, video and/or audio output 214 of the device at the end of each session, or upon a user's request.

Display device 210 may further comprise an image processing element, provided for analyzing the imagery data recorded by optical sensor 211. Such analysis may include selecting highlight images, i.e. at least one peak performance image which pertains to at least one selected feature from the performance information gathered for the session. Video content may be easily available for wireless communication and social sharing, and such sharing may be even encouraged by the interface of the application. The photo and image records are preferably saved in local storage means 212 of display device 210, and access to the images is available through the application or through a general image library built-in in display device 210. Default image quality are set by the application, but can be changed by the user according to his preferences. Video images may be recorded with various performance information data imprinted over it, and may be further subjected to additions in the form of visual and/or audio and/or text overlays, editing the video, editing of several video images into a summary version, etc.

In a preferred embodiment of the present invention, display device 210 is further provided with communication means enabling its connection to server 220, which could be a computer server, a cloud-like server or a main computational center. Server 220 preferably comprises storage means 222 and processor 223. In such embodiments, data and performance information from display device 210 are transmitted to server 220 and are subjected to further analysis to obtain performance information. The results and analysis may then be transmitted back to display device 210 to be displayed on video, image and/or audio means 214 of the device. Server 220 may also collect broad statistical information including data of all the users of the system, performance of the system and the infants practicing Tummy Time exercise using the system. Statistics determined by server 220 may lead to a baseline of normal infant development and further establish a standard of care setting a threshold above which a medical condition is suspected.

In a preferred embodiment of the present invention, the analysis and results may be transmitted directly from the device or through the cloud based server, or any other server, to any third party 230, preferably a health or medical facility, service or personnel, comprising storage unit 232 for containing the data, processor 233 for any further analysis and display means 234 for presenting the data to medical personnel.

In preferred embodiments, data may be sent back to the device from server 220 and/or from third party 230 (directly or indirectly). This data may comprise additional analysis of previously sent data and performance information, statistics of other users of the application, direction or recommendation for infant's future practicing, invitation for consultation, a trigger for the system to configure the practice session in a specific mode based on the infant's medical condition, or any medical/health/infant related content.

According to some embodiments, an input and analysis unit provides accumulative analysis of the performance information and either records the data, presents it to the user or transmits it further to external servers or medical centers or health or medical service or personnel, such as 220 or 230.

In some embodiments indication of abnormal performance information may trigger an alarm or notification of the condition on the display device or on computer center 230 used by the medical service or personnel, or on display device 210, or in server 220 or any combination thereof.

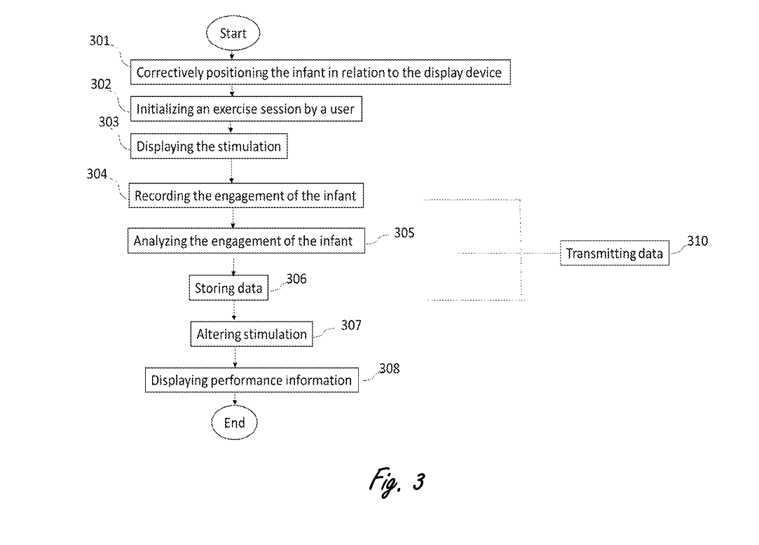

Reference is now made to FIG. 3, illustrating a high level overview of the method provided by the present invention. The method recites a few main steps after opening the application by a user. After correct positioning the infant in relation to the display device (301), the user can start an exercise session (302). Then the display device displays visual or audiovisual stimulation (303). The engagement of the infant is recorded (304). In the meanwhile, the infant's engagement is analyzed (305). The step further includes identification of the infant's face and gaze orientation and position. This data is analyzed either locally in the device and/or in a remote processor, resulting in performance information. Step 306 includes storing the raw data provided by the sensors in a storage means. At this stage, the raw data may be transmitted to other devices and computers, or servers. Alteration of the video and/or audio output 307 is then executed according to predetermined protocols corresponding to various outcomes detected in the performance information, thus providing real-time stimulation feedback to the infant's behavior. According to some preferred embodiments, alteration of the stimulation starts a few seconds after initiation of an exercise session. In preferred embodiments, once the application's session is stopped, at least part of the current session's performance information is presented on a display means for it to be reviewed by the user (usually a parent). The summary of the performance information displayed may include the most recent exercise session, and/or comparison between the most recent session to past sessions, and/or an average of any number of past sessions. Data may be transmitted to another computer 310, at any time or step. In some embodiments, data may be configured to be sent automatically. In other preferred embodiments, data transmitted from the device undergoes further analysis and may be compared to cumulative averages of performance information collected from peer groups, mainly for basic research and diagnosis of various medical conditions pertaining to abnormal performance information. The analyzed data or any cumulative data may be sent back to the device and may be saved in the device's storage means, displayed in the device, or available for access by the user of the device.

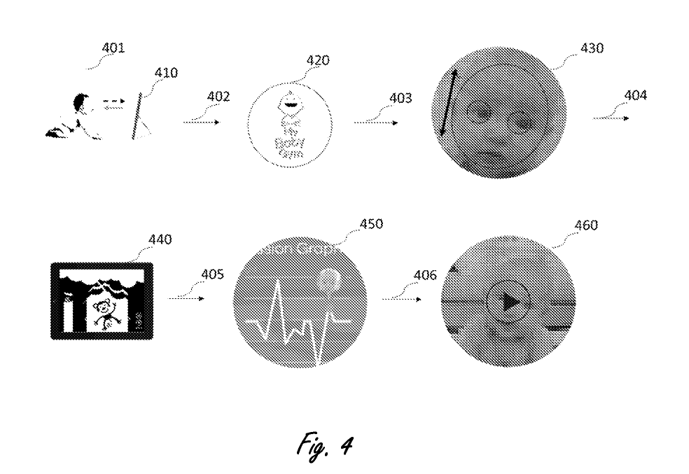

Reference is now made to FIG. 4, which schematically presents an overview of an exercise session according to a preferred embodiment of the systems provided by the present invention. The exercise session starts with positioning an infant in front of a tablet 410 comprising an optical sensor. The application 420 installed in the tablet guides a user of the application to position the infant correctly in front of the tablet and/or to position the tablet correctly in front of the infant. After positioning the infant correctly (401), then the user can initiate an exercise session by start the application 420. The infant's face position and gaze 430 are detected and tracked. The stimulation 440 (such as visual, audio, vocalization or audiovisual stimulation) is provided. Tracking and providing stimulation are carried out simultaneously. Audiovisual stimulation 440 can be altered or personalized according to the features of particular infant (step 403 and 404). Performance of the infant during the exercise session is recorded, measured and analyzed by the application 420. Audiovisual stimulation 440 is then altered according to the predefined protocol that correspond to the session's performance information. The analysis of exercise session and performance information 450, such as duration of exercise session, time of exercise, number of roll ups, highest point reached by the infant, quantitative improvement, overall progress, emotions during the exercise session et al., can be viewed on the display device. Images and video from the exercise session 460 and analysis of the exercise session 450 are stored in the tablet or cloud storage service. The data of the exercise session (450 and 460) can then be shared with family members and friends, reviewed by the health professionals or other users of the application 420.

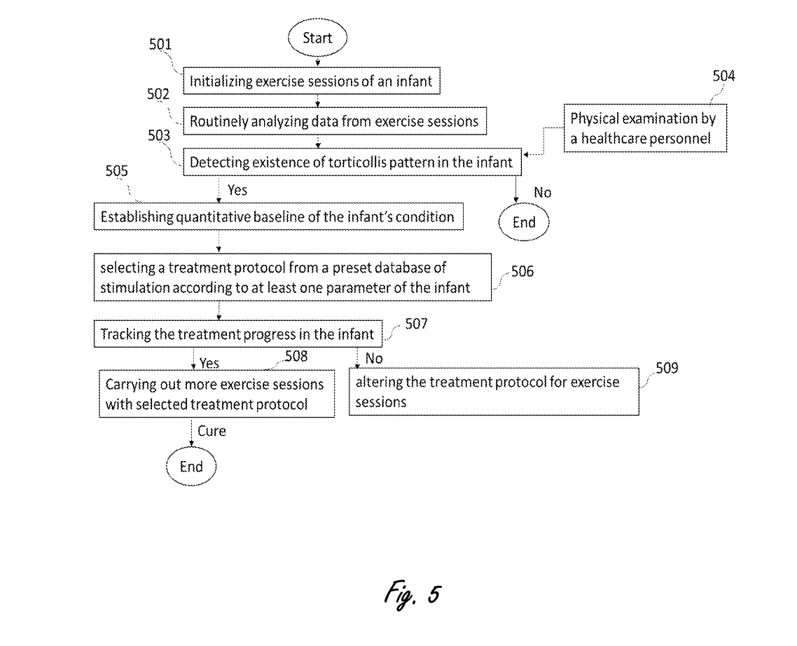

Reference is now made to FIG. 5, which presents a high level flow chart of a preferred embodiment of the method of treating torticollis disclosed in the present invention. The method recites a few main steps after initiation of exercise sessions of an infant by a user 501 through the application installed in a device. Following initiation exercise sessions, details as described in FIG. 4, the next step 502 the raw data obtained during the exercise session are routinely analyzed locally in the device or in a remote processor. The diagnosis could also be performed during a physical examination by a healthcare personnel (504). If there is no torticollis pattern, then the method can be terminated. When a torticollis pattern is detected from tracking the infant's face posture throughout exercise session(s), then a diagnostic alert will be sent to the health personnel that follows this infant, for further inquiry. Based on the analysis data from exercise sessions, the system then establishes a quantitative base line of the infant's condition, such as type and amplitude of the tilt, rotation or flexion. A personalized protocol for exercise session for treatment of torticollis in the infant is then selected from a preset database of stimulations according to at least one parameters of the infant (506). The preset database of stimulation comprises a series of visual stimulations. According to some preferred embodiments, the visual stimulation is accompanied by audio stimulation and/or vocalization. The personalized treatment protocol motivates the infant to bring his or her head into a symmetrical position by exposing the infant to selected stimulation. The selection of the treatment protocol is based on the established infant's condition. The selection of the treatment protocol is carried out according to at least one parameter of the infant including the infant's age, medical symptom of the infant, the infant's performance information, a healthcare personnel's decision, a user's choice, treatment progress of the infant or any combination thereof. Medical symptoms of the infants include but are not limited to type of torticollis, direction of head position (i.e. left or right tendency), amplitude of tilt, amplitude of rotation and amplitude of flexion. For example, if the infant's head tends to be in the right side leading to the gaze directed to the left side, the visual stimulation is then focused in the right area of the display device to motivate the infant to move into a symmetrical position. The system also allows the healthcare personnel to create his or her own preferred protocol, where he or she can define various parameters manually. The system monitors the infant's progress in every exercise session (507). In some preferred embodiments, the stimulation can adjust as a function of treatment progress to allow a gradual scaling and ensure the infant to experience successful effort. If there is no progress observed, then treatment protocol will be altered according to at least one parameter of the infant (509). More exercise sessions will be carried out until the cure of torticollis in the infant or achieving a preset goal. The system enables healthcare personnel to track the treatment progress remotely. Performance information and videos from exercise sessions are then stored locally and/or send to an electronic device selected from the group consisting of a network, a remote server, a cloud-like server, a remote computer, a remote display means or any combination thereof, where they can be reviewed by a healthcare personnel or family members of the infant. Furthermore, digital information stored or processed in the remote electronic device (e.g. analyzed performance information, user instructions, computer readable instructions et al) as mentioned before can be transmitted back to the display means. The system also delivers automatic updates that contain the treatment progress data to the infant's healthcare personnel or family members.

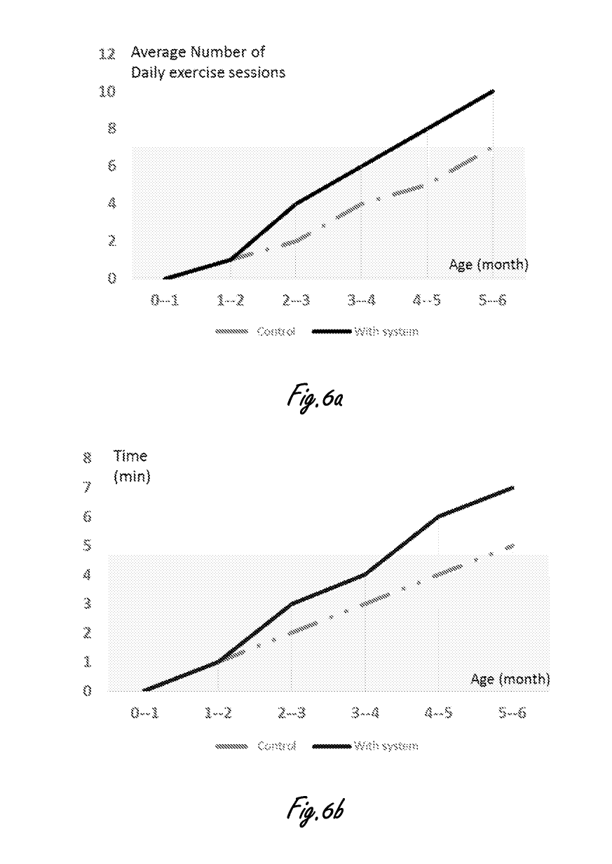

Reference is now made to FIG. 6, which presents statistical results (n=36) of impact of using the disclosed system to encourage Tummy time exercise in infants. Following initial use of the disclosed system by a group of infants (n=18) and their parents during beta test and comparing their usage data and their parent's usage reporting to a control group (i.e. the group not using the disclosed system, n=18), there have seen a clear improvement in beta-test parents' compliance and exercise duration compared to the control group. The single most important factor for parent's compliance in practicing Tummy Time is their perception of the baby's experience. When the baby cries, parents tend to slower practice initiation in order to avoid upsetting the baby. When the baby doesn't cry, they perceived the practice as a strengthening activity and tend to increase its initiation. During the beta test, the disclosed system has shown to create a substantial increase in the average number of sessions that are initiated by the parents compared to the control group as presented in FIG. 6a. As the infants practice more Tummy Time, it become easier for them to lift head and stay in the target position for a longer period of time. During the beta test, the disclosed system has shown to create a substantial increase in the average duration of a single exercise session as illustrated in FIG. 6b.

### Example 1: Exercise Session and Interface

An exercise session is composed of a series of interactive movies displayed continuously. The movies and content (audiovisual stimulation) is selected by the user (usually the parent or healthcare personnel) from the menu of available and relevant contents in the application. The interface may recommends the user of appropriate movies and content according the developmental age of the infant, diagnosed medical conditions (such as Torticollis) and according to past performance information of the infant with respect to the stimulation.

An exercise session comprises:
a. Activating a computer executable program by pressing a “perform exercise” link.
b. Choosing video content and voicing stimulation.
c. Positioning of the infant in front of the display screen. The interface presents a graphic guide for the user that recommends on the infant's most efficient practice position. It also guiding the user to position the optical sensor in a way that will allow the system to optimally perform both the face and gaze recognition and tracking as well as capture and save a session video that presents the infant in the center of the image.
d. In order to start the session, the user needs to press on a “start” function.

An exercise session may end in a few possible conditions:
a. User pressing the “end” link.
b. An internal timer is set to end the application following a predetermined duration of time (i.e. after 1, 3, 5 minutes etc.)
c. The optical sensor and processing unit are unable to detect an infant's face and gaze for a specified amount of time (e.g. 30 seconds), and no response is being recorded to the notifying message that is presented to the user is being for another period of time (e.g. 60 seconds).

### Example 2: Video and/or Audio Content Presented to the Infant

At each exercise session the infant is subject to three kinds of stimulation: visual stimulation, preferably in the form of some animation; audiovisual stimulation (i.e. audio stimulation accompanied with the visual stimulation), preferably in the form of the animation with music used as a soundtrack in various parts; and audio stimulation pertaining to the performance information received by analyzing data of the infant's engagement with the display device, preferably in the form of vocalizations. The content of the stimulation is determined by a user (e.g. parents, a caretaker) or by predetermined protocols taking into consideration of parameters such as the infant's age, progress of the infant, the infant's performance information, infant's medical conditions, previous exercise session et al. Each content contains a combination of the three stimulation. For each exercise session, the user choose a number of (e.g. four) audiovisual content from the content gallery and one type of vocalization stimulation to formulate the stimulation for the entire exercise session. The algorithm of presenting the content of stimulation takes into consideration the head orientation and gaze tracking, pre-diagnosed medical condition, as well as other factors such as the infant's age, progress in the past, engagement time in the session, user preferences, etc.

### Example 3: States and Timings of the Application

The predetermined protocols for setting stimulation types is also subjected to a basic instructions algorithm determining general operating features, such as minimal transition times between different stimulations, various alerts and notifications regarding the usage of the display device when the system no longer detects a participant, or when performing basic operations such as starting the application, ending it or using any of the standard features set by an application. For example, the basic algorithm sets a minimal time period for transitioning between different visual animations contents, in order to provide the infant with an enjoyable experience and avoid over-stimulation. Another example would be predetermined time frame for detecting facial recognition after losing one, after which if no recurring recognition is provided, the system will terminate the session.

### Example 4: Audio Stimulation

During audio stimulation, preset or custom-made vocal recordings are being vocalized to the infant at various points throughout an exercise session. The main feedback voices refer to general contact with the baby, focusing the baby's attention on the screen and encouraging the baby on his progress. Audio voicing can be executed by three different options: (1) language, i.e. words the baby may recognize; (2) sound, i.e. various attention grabbing vocalizations; (3) personal vocal recordings by the user (i.e. parent, caregiver).

### Example 5: Torticollis Treatment

The system disclosed in the present invention is useful in treating various medical conditions, in particular, torticollis. Torticollis is a popular physical problem that affects infants. It is an abnormal, asymmetrical head or neck position that is expressed by a fixed or dynamic tilt, rotation, or flexion of the head and/or neck. The basic treatment for this problem includes guiding parents by a healthcare personnel in ways to promote symmetry of the infant's neck in his daily routines and activities. It also includes a recommendation to practice lots of Tummy Time exercise in order to strengthen the neck and back muscles and support the efforts to enlarge the infant's span of movement. The system disclosed in the present invention for treating torticollis is to encourage the Tummy Time exercise in a manner that corrects detected torticollis symptoms in the infant. This is done by exposing the infant to stimulation that is designed to increase his or her motivation to reach a symmetrical head position while practice Tummy Time. The torticollis treatment process includes three main steps: diagnostics, treatment exercise and progress tracking.

Infant's regular exercise session data is routinely analyzed by the system. This analysis is tunes to find, among other things, indication for torticollis, by analyzing the data of the infant's face posture throughout the session. When the system detects a torticollis pattern, it sends a diagnostic alert to the healthcare personnel that follows this infant, for further inquiry. It also recommends a specific practice protocol as a proposed treatment. Alternatively, the infant can be diagnosed with torticollis by his doctor or physiotherapist during a physical examination. Following diagnosis, the system creates a quantitative base line for this patient condition (type and size of the tilt/rotation/flexion). Based on that, the system recommends a specific exercise protocol for the infant's proposed treatment.

The exercise protocols expose the infant to stimulation that is designed to motivate him or her to bring the head into a symmetrical position. In a typical session, the infant is put to practice Tummy Time with the system, similar as in a regular practice session. The major difference is that the content the infant is exposed to is designed specifically to deal with torticollis cases, and enable these patients a customized treatment session. This personal customization is based on the infant diagnosis as it is recorded and defined by the system. The customization or personalization is done according to various aspects as elaborated below:
a. developmental age of the infant;
b. infant's medical condition—customizing the content stimulation to the direction of head, the amplitude of tilt, rotation and/or flexion that needs to be compensated; the content is focused in certain areas of the screen, specially designed to motivate the infant to move into a head symmetry position;
c. treatment process—the stimulation changes as a function of the treatment progress, to allow a gradual scaling and ensure that the infant will be able to experience successful efforts;
d. health professional preferred protocol—the system produces a customized exercise protocol based on the infant diagnosis as it is was recorded and defined by the system. At the same time, it allows the health professional to create his or her own preferred protocol, where he or she can define the various parameters manually. In this way, the infant can practice the system's recommended protocol, the health professional preferred protocol or any combination thereof.

The system monitors the infant's progress in the relevant parameters in every training session. It then enable healthcare personnel a remote access to this data as well as the session's video recording, so they can review and track the treatment progress. The system also delivers automatic updates that contain the treatment progress data to the infant's healthcare personnel.

While the invention is susceptible to various modifications and alternative forms, specific embodiments thereof have been shown by way of example in the drawings and the above detailed description. It should be understood, however, that it is not intended to limit the invention to the particular forms disclosed, but on the contrary, the intention is to cover all modifications, equivalents, and alternatives falling within the spirit and scope of the invention as defined by the appended claims.

1. A method for monitoring and encouraging exercise of an infant, characterized by steps of:
a. providing guidance for positioning an optical sensor configured to record video of said infant in front of said infant or vice versa, in at least on form elected from the group consisting of a silhouette of an infant, text instructions, visual instructions, audio instructions, color coded indicator and any combination thereof;
b. displaying first audiovisual stimulation adapted for encouraging said infant to exercise on a display means configured to be substantially flat and placed in front of said infant;
c. recording engagement of said infant by said optical sensor;
d. monitoring and analyzing in real-time said engagement of said infant by a processor, thereby obtaining performance information;
e. altering said first audiovisual stimulation to second audiovisual stimulation adapted for reengagement of said infant when performance information indicates conditions selected from the group consisting of: no engagement of said infant, the engagement of said infant for a predetermined period of time, said infant's head, chest or hands in predefined postures or positions, a predefined gaze pattern, a predefined activity of said infant, a predefined reaction of said infant, progress of said infant or any combination thereof;
f. returning to said first audiovisual stimulation when said infant is engaged; and
g. storing said performance information on a storage means.

2. The method according to claim 1,
wherein said processor and storage means are located in proximity to said optical sensor, or in a remote location.

3. The method according to claim 1, wherein said step of recording and said step of monitoring and analyzing are performed simultaneously.

4. The method according to claim 1, wherein said step of recording and said step of monitoring and analyzing start before said step of displaying visual stimulation.

5. The method according to claim 1, further comprising the step of producing vocalizations when said performance information indicates condition selected from the group consisting of: no engagement of said infant, the engagement of said infant for a predetermined period of time, said infant's head, chest or hands in predefined postures or positions, a predefined gaze pattern, a predefined activity of said infant, a predefined reaction of said infant, progress of said infant or any combination thereof.

6. The method according to claim 1, further comprising the step of altering said audiovisual stimulation or vocalization in accordance with parameters selected from the group consisting of said infant's age, said infant's performance, progress of said infant, duration of a usage of said system, a user's choice, diagnosed medical condition of said infant, said infant's head, chest or hands in predefined postures or positions, a predefined gaze pattern, a predefined activity of said infant, a predefined reaction of said infant and any combination thereof.

7. The method according to claim 1, further comprising the step of recommending said vocalization and/or audiovisual stimulation in accordance with parameters selected from the group consisting of said infant's age, said infant's performance, progress of said infant, duration of a usage of said system, a user's choice, diagnosed medical condition of said infant, said infant's head, chest or hands in predefined postures or positions, a predefined gaze pattern, a predefined activity of said infant, a predefined reaction of said infant and any combination thereof.

8. The method according to claim 1, further comprising the step of automatically stopping said audiovisual stimulation after sensing an outcome selected from the group consisting of inability to detect face or gaze of said infant, losing tracking of said face or gaze, use of said stimulation for a specified amount of time, a predefined activity of said infant, a predefined reaction of said infant and any combination thereof.

9. The method according to claim 1, further comprising the step of stopping said audiovisual stimulation manually.

10. The method according to claim 1, wherein said display means is selected from the group consisting of a mobile phone, a tablet, a laptop, an e-reader, a digital wall, a smart TV, a computer, and any combination thereof.

11. The method according to claim 1, further comprising the step of analyzing cumulative performance information at a predetermined frequency.

12. The method according to claim 1, further comprising the step of selecting said features of said performance information from the group consisting of duration of an exercise session, height of said infant's face, highest height reached during exercise, number of roll ups, duration of said engagement with said stimulation, comparison of current usage performance information with past usage performance information, comparison of current usage performance information with at least one other user's performance information, comparison of current usage performance information with cumulative peer group performance information, said infant's gaze correlation with said visual stimulation, engagement steadiness, the amount of time spent in each position of said infant, said infant's reflected mood, said infant's physical steadiness, said infant's hand position, body posture development level of said infant, the amount of time spent in each position of the infant's head, measured angle of said infant's eyes in relation to nose, measured angle of said infant's eyes in relation to the horizon, information extracted from skin tone of said infant and any combination thereof.

13. The method according to claim 1, further comprising the step of detecting abnormal performance information relating to a medical conditions selected from the group consisting of abnormal or inability of said infant to track said visual stimulation, tendency of said infant to bias his engagement from a specific direction, significant deviance from said self-cumulative past performance information, significant deviance from said peer group-cumulative past performance information, abnormal orientation detection of said infant's head, strabismus, cataract, lazy eye, apathy, Torticollis, abnormal orientation of said infant's body posture, hypotonia, hypertonia, anemia, skin rash, arrhythmia and any combination thereof.

14. The method according to claim 13, further comprising the step of providing an alert following detection of said abnormal performance information.

15. The method according to claim 1, wherein a predetermined time limit is imposed to a single exercise session.

16. The method according to claim 1, wherein a predetermined time limit is imposed to daily exercise sessions.

17. The method according to claim 1, further comprising the step of choosing an exercise session to be archived or not by a user.

18. The method according to claim 1, wherein said video recording results in video of at least part of an exercise session stored in said storage means, thereby obtaining at least one archive video.

19. The method according to claim 18, further comprising the step of processing at least part of said archive videos of said infant by an image processing element, selecting at least one peak performance image corresponding to at least one of said selected features recognized by said performance information and displaying said peak performance image on a display means.

20. The method according to claim 1, further comprising the step of graphically presenting at least part of said performance information and/or said archive video on said display means at the end of an exercise session.

21. The method according to claim 19, further comprising the step of electronically sharing with at least one other user of said display means the content selected from the group consisting of said archive videos, performance information, said at least one peak performance image and any combination thereof.

22. The method according to claim 18, further comprising the step of electronic sharing via uploading the content to websites or social sharing platforms.

23. The method according to claim 21, further comprising the step of manually or automatically performing on said archive videos at least an action prior to said step of electronically sharing, said action consisting the group of editing, adding data, adding text, overlaying at least one visual feature, overlaying at least one audio feature, adding at least one peak performance image, editing a plurality of videos into a single video and any combination thereof.

24. The method according to claim 19, further comprising the step of digitally transmitting at least part of said video archive, said performance information and/or said peak performance images to an electronic device selected from the group consisting of a remote server, a cloud-like server, a remote computer, a remote display device and any combination thereof, and optionally digitally transmitting from said electronic device, back to said display device, analyzed performance information, user instruction or any computer readable instructions pertaining to said performance information.

25. The method according to claim 1, further comprising the step of transmitting said video archive and/or said performance information to a medical facility or personnel, and optionally digitally transmitting from said medical facility or personnel, back to said display device, analyzed performance information, user instructions or any computer readable instructions pertaining to said performance information.

26. The method according to claim 25, wherein said medical facility is selected from the group consisting of a family doctor, a pediatrics doctor, an orthopedic, a neurologist, an ophthalmologist, a physiotherapist, a nurse, a health professional, a medical information center, a telehealth or telemedicine service and any combination thereof.

27. A computer executable program adapted to perform a method as claimed in claim 1 when running on a computer, a network or a server system.

28. A computer-readable storage medium, having encoded thereon said computer program according to claim 27.

29. A system for monitoring and encouraging exercise of an infant comprising:
a. an optical sensor configured to detect and video record engagement of said infant;
b. a processor configured to monitor and analyze in real-time said engagement of said infant for obtaining performance information;
c. a storage means for storing said performance information;
d. an audio means;
e. a display means configured to be substantially flat and display audiovisual stimulation corresponding to a protocol predetermined by selected features recognized by said performance information;
wherein said processor is configured to provide guidance for positioning said optical sensor in front of said infant or vice versa, in at least one form selected from the group consisting of a silhouette of an infant, text instructions, visual instructions, audio instructions, color coded indicator and any combination thereof; and
further wherein said processor is configured to cause said display means to alter said audiovisual stimulation adapted for reengagement of said infant when said performance information indicates condition selected from the group consisting of no engagement of said infant, the engagement of said infant for a predetermined period of time, said infant's head, chest or hands in predefined postures or positions, a predefined gaze pattern, a predefined activity of said infant, a predefined reaction of said infant, progress of said infant or any combination thereof.

30. The system according to claim 29, wherein said processor is located in proximity to said optical sensor, or in a remote location.

31. The system according to claim 29, wherein said storage means is located in proximity to said optical sensor, or in a remote location.

32. The system according to claim 29, wherein said processor is configured to cause said audio means to produce vocalizations when said performance information indicates condition selected from the group consisting of: no engagement of said infant, the engagement of said infant for a predetermined period of time, said infant's head, chest or hands in predefined postures or positions, a predefined gaze pattern, a predefined activity of said infant, a predefined reaction of said infant, progress of said infant or any combination thereof.

33. The system according to claim 29, wherein said audiovisual stimulation or vocalization is altered in accordance to parameters selected from the group consisting of said infant's age, said infant's performance information, said infant's progress, duration of a usage of the system, a user's choice, a predefined activity of said infant, a predefined reaction of said infant, diagnosed medical condition of said infant and any combination thereof.

34. The system according to claim 29, wherein said system recommends said audiovisual stimulation and vocalization in accordance with parameters selected from the group consisting of said infant's age, said infant's performance information, said infant's progress, duration of a usage of the system, a user's choice, a predefined activity of said infant, a predefined reaction of said infant, diagnosed medical condition of said infant and any combination thereof.

35. The system according to claim 29, wherein said processor is configured to automatically stop said stimulation after sensing an outcome selected from the group consisting of inability to detect face or gaze of said infant, losing tracking of face or gaze of said infant, use of said stimulation for a predefined amount of time, a predefined reaction of said infant and any combination thereof.

36. The system according to claim 29, wherein said stimulation is stopped manually by a user.

37. The system according to claim 29, wherein said processor is configured to provide at the end of an exercise session, on said display means, graphical presentation selected from the group consisting of at least part of said performance information, archive video and at least one peak performance image and any combination thereof.

38. The system according to claim 29, wherein said display means is selected from the group consisting of a mobile phone, a tablet, a laptop, an e-reader, a smart TV and a personal computer, a digital wall, and any combination thereof.

39. The system according to claim 29, wherein a predetermined time limit is imposed to a single exercise session.

40. The system according to claim 29, wherein a predetermined time limit is imposed to daily exercise sessions.

41. The system according to claim 29, wherein a user chooses an exercise session to be archived or not.

42. The system according to claim 29, wherein said processor is further configured to analyze cumulative performance information in a predetermined frequency.

43. The system according to claim 29, wherein said selected features recognized by said performance information are selected from the group consisting of duration of an exercise session, height of said infant's face, highest height reached during exercise, number of roll ups, duration of said engagement with said stimulation, comparison of current usage performance information with past usage performance information, comparison of current usage performance information with at least one other user's performance information, comparison of current usage performance information with cumulative self or group's performance information, said infant's gaze correlation with said visual stimulation, engagement steadiness, the amount of time spent in each position of said infant, said infant's reflected mood, said infant's hand position, body posture development level of said infant, the amount of time spent in each position of said infant's head, measured angle of said infant's eyes in relation to nose, measured angle of said infant eyes in relation to the horizon, information extracted from skin tone of said infant and any combination thereof.

44. The system according to claim 29, wherein said processor is configured to detect abnormal performance information relating to a performance selected from the group consisting of abnormal or inability of said infant to track said visual stimulation, tendency of said infant to bias his engagement from a specific direction, significant deviance from said self-cumulative past performance information, significant deviance from said peer group-cumulative past performance information, abnormal orientation detection of said infant's head, strabismus, cataract, lazy eye, apathy, torticollis, abnormal orientation of said infant's body posture, hypotonia, hypertonia, anemia, skin rash, arrhythmia and any combination thereof.

45. The system according to claim 44, wherein said processor is configured to provide an alert following detection of said abnormal performance information.

46. The system according to claim 29, wherein said storage means is configured to record said engagement in at least part of an exercise session resulting in at least one archive video.

47. The system according to claim 29, further comprising an image processing element configured to process capturing of said engagement of said infant, select at least one peak performance image corresponding to at least one of said selected features recognized by said performance information and display at least one said peak performance image on said display means.

48. The system according to claim 47, wherein said system is configured to encourage a user to electronically share with at least one other user of said display means content selected from the group consisting of performance information, said archive video, said at least one peak performance image and any combination thereof.

49. The system according to claim 48, wherein said processor is configured to automatically or manually perform on said archive videos action consisting the group of editing, overlaying at least one visual feature, overlaying at least one audio feature, adding at least one peak performance image, editing a plurality of videos into a single video and any combination thereof.

50. The system according to claim 29, further comprising communication means configured to electronically transmit at least part of said performance information, said archived video and/or said at least one peak performance image to an electronic device selected from the group consisting of a network, a remote server, a cloud-like server, a remote computer, a remote display means and any combination thereof, and optionally said electronic device comprises communication means configured to electronically transmit back to said device analyzed performance information, user instructions or any computer readable instructions pertaining to said performance information.

51. The system according to claim 50, wherein said electronic device is located in a medical facility, and optionally said medical facility comprises communication means configured to electronically transmit back to said electronic device analyzed performance information, user instructions or any computer readable instructions pertaining to said performance information.

52. The system according to claim 51, wherein said medical facility is selected from the group consisting of a family doctor, a pediatrics doctor, an orthopedic, a neurologist, an ophthalmologist, a physiotherapist, a nurse, a healthcare personnel, a medical information center, a telehealth or telemedicine service and any combination thereof.

53. Non-transitory, tangible, computer-readable storage media containing a program of instructions containing algorithms configured to cause a computer processing system running the program of instructions to monitor and encourage exercise of an infant by:
a. providing guidance to correctly positioning an optical sensor configured to record video of said infant in front of said infant or vice versa, in at least one form selected from the group consisting of a silhouette of an infant, text instructions, visual instructions, audio instructions, color coded indicator and any combination thereof;
b. displaying audiovisual stimulation on a display means configured to be substantially flat and placed in front of said infant;
c. recording engagement of said infant by said optical sensor;
d. monitoring and analyzing in real-time said engagement of said infant by said processor, thereby obtaining performance information;
e. altering said audiovisual stimulation to reengagement of said infant when said performance information indicates condition selected from the group consisting of: no engagement of said infant, the engagement of said infant for a predetermined period of time, said infant's head, chest or hands in predefined postures or positions, a predefined gaze pattern, a predefined activity of said infant, a predefined reaction of said infant, and
f. storing said performance information on a storage means.

54. A method of diagnosing and treating torticollis in an infant, said method is characterized by steps of:
a. providing a system for monitoring and encouraging exercise of an infant comprising:
i. an optical sensor configured to detect and video record engagement of said infant;
ii. a processor configured to monitor and analyze in real-time said engagement of said infant for obtaining performance information;
iii. a storage means for storing said performance information;
iv. an audio means; and
v. a display means configured to be substantially flat and display audiovisual simulation corresponding to a protocol predetermined by selected features recognized by said performance information;
b. analyzing performance information from by said processor from at least one exercise session of said infant;
c. detecting torticollis pattern in said infant via said optical sensor of said system;
d. establishing quantitative baseline of said infant's medical symptom based on analysis of said at least one exercise session.

55. The method according to claim 54, further comprising steps of selecting a protocol from a preset database of stimulation for said infant according to at least one parameter of said infant and tracking treatment progress of said infant.

56. The method according to claim 54, wherein said step of selecting is based on said at least one parameter of said infant selected from the group consisting of said infant's age, medical symptom of said infant, said infant's performance information, a healthcare personnel's decision, a user's choice, treatment progress of said infant and any combination thereof.

57. The method according to claim 54, wherein said medical symptom of said infant is selected from the group consisting of type of torticollis, amplitude of tilt, amplitude of rotation, amplitude of flexion and any combination thereof.

58. The method according to claim 54, further comprising the step of alerting diagnosis of torticollis in said infant to relevant healthcare personnel after said step of detecting torticollis pattern.

59. The method according to claim 54, wherein diagnosis of torticollis is performed by a healthcare personnel.

60. The method according to claim 59, wherein said healthcare personnel is selected from the group consisting of a physician, a nurse, a physician assistant, a nursing assistant, an attendant, a technician, a physiotherapist, a contractual staff not employed by a health-care facility, a telemedicine service and any combination thereof.

61. The method according to claim 54, wherein said preset database of stimulation comprises a series of visual stimulation.

62. The method according to claim 61, further comprising the step of providing audio stimulation to accompany said visual stimulation, thereby providing audiovisual stimulation.

63. The method according to claim 61, further comprising the step of providing vocalization as a form of stimulation.

64. The method according to claim wherein said visual, audio, audiovisual stimulation or vocalization is designed to adjust focus and/or content of said stimulation in a display means according to said at least one parameter of said infant.

65. The method according to claim 54, further comprising the step of manually changing protocol by a healthcare personnel.

66. The method according to claim 54, wherein said method promotes parent of said infant to compliant with daily exercise requirement.

67. The method according to claim 54, further comprising the step of digitally transmitting the performance information, archived videos and peak performance images from exercise sessions and/or the progress of said infant to an electronic device selected from the group consisting of a server, a network, a cloud-like server, a computer, a display means and any combination thereof, and optionally digitally transmitting, from said electronic device back to said display means, analyzed performance information, user instructions or any computer readable instructions pertaining to the performance information.

68. The method according to claim 60, further comprising the step of remotely accessing said data of exercise sessions and/or said treatment progress by said healthcare personnel.

69. The method according to claim 54, further comprising the step of sending automatic update of progress of said infant to relevant healthcare personnel or at least one family member.

70-78. (canceled)
